(19) United States
(12) Patent Application Publication
(10) Pub. No.: US 2005/0075550 A1
(43) Pub. Date: Apr. 7, 2005

(54) Quick-clip sensor holder

(76) Inventor: Eric W. Lindekugel, Fort Collins, CO (US)

Correspondence Address: MARSH FISCHMANN & BREYFOGLE LLP, Suite 411, 3151 South Vaughn Way, Aurora, CO, 80014, US

(21) Appl. No.: 10/679,129

(22) Filed: Oct. 3, 2003

(51) Int. Cl. A61B 5/00

(52) U.S. Cl. 600/344

(57) ABSTRACT

The present invention provides a clip-type medical sensor holder for use in positioning a pulse oximetry sensor relative to a patient appendage. In particular, a clip-type sensor holder is provided that clips around a portion of a patient appendage without necessarily applying a clamping force to that appendage to reduce or eliminate vasoconstrication. The sensor holder includes first and second opposing members in an opposing and hinged relationship that allows these members to move from an open position to a closed position relative to one another. When in the closed position, these opposing members are sized to receive a patient appendage (e.g., a finger) therebetween. A resilient member interconnected to each of the members maintains the sensor holder in the closed position about the patient appendage. In one embodiment, the sensor clip is formed as a one-piece unit allowing simplified construction and a reduced part count.

### FIELD OF THE INVENTION

The present invention is generally directed to a holder for positioning a medical sensor relative to patient tissue. More specifically, the present invention is directed to a clip-type sensor holder for use in positioning a pulse oximetry sensor relative to a patient appendage.

### BACKGROUND

In many medical applications it is desirable to hold one or more sensors in contact with a patient's tissue such that various non-invasive measurements of physiological parameters may be made. For example, a common technique used to monitor blood analyte concentrations and blood oxygen levels is pulse oximetry. In this regard, it is known that the light transmissivity and color of blood is a function of the oxygen saturation of the heme in the blood's hemoglobin. For example, heme saturated with oxygen appears bright red because saturated heme is relatively permeable to red light. In contrast, deoxygenated heme appears dark and bluish, as it is less permeable to red light. A pulse oximeter system measures the oxygen content of arterial blood by utilizing a pulse oximetry sensor to illuminate the blood with, for example, red and infrared radiation and determine the corresponding amounts of red and infrared radiation that are absorbed by the heme in the blood. In turn, such light absorption amounts may be employed by a pulse oximetry monitor in conjunction with known calibration information to determine blood oxygen levels and/or other blood analyte concentrations.

As may be appreciated, in order to accurately compute blood analyte concentrations, it is important that the pulse oximetry sensor be properly oriented relative to a patient's tissue. In this regard, it will be noted that pulse oximetry sensors generally include one or more light emitters and detectors that are typically held in direct contact with the tissue of a patient such that they may emit and detect light relative to that tissue. Holding the sensors too loosely relative to the tissue may result in erroneous readings or sensor failure. In contrast, sensors held too tightly to the tissue may interfere with the physiological parameter(s) they are supposed to monitor. That is, if a sensor is pressed into the tissue, vasoconstriction, or, pressure necrosis may result wherein blood flow through the tissue is reduced. Reduction of blood flow may in turn affect any subsequent calculations of blood analyte concentrations.

A number of means are utilized for holding the emitters and detectors of a pulse oximetry sensor in contact with a patient's tissue; two common types are adhesive-type and clip-type sensor holders. Adhesive-type sensor holders, as their name suggests, utilize an adhesive to bond a sensor to a patient's tissue. Adhesive-type holders are well suited for medical procedures where a sensor will remain attached to a patient for an extended period. Clip-type sensor holders allow for ready application and removal of a sensor to a patient. Accordingly, clip-type sensor holders are often preferred for spot-checking blood oxygen levels. Clip-type sensor holders typically include two hingedly connected housings that releaseably clip onto an end portion of a patient appendage (e.g., finger, ear lobe, nasal septum, etc.). The action of a spring typically holds the clip-type sensor holder on the patient appendage by compressing the housings onto the patient appendage. To prevent excess pressure on the appendage, these springs typically exert little force. As a result, these clip-type sensors tend to readily fall off patient appendages. Furthermore, the use of a clip-type sensor holder may be limited to a narrow range of appendage sizes. For example, a small finger may not displace the spring sufficiently to maintain the clip on the finger while a larger finger may displace the spring such that excessive force is applied to the finger.

There are a number of additional considerations for sensor holders. First, the holders should have the ability to achieve a reliable interface between the sensor and the patient's tissue. Inherent in this first consideration is the need to securely hold a sensor in place relative to the patient's tissue in a manner that is resistant to unintended removal and/or slippage relative to the tissue. Second, the holder and sensor should be adapted for ready application and removal from the patient with minimal patient discomfort and ease of use for the applicator. Finally, the holder and sensor should provide a gentle interface with the patient's skin.

### SUMMARY OF THE INVENTION

In view of the foregoing, a primary objective of the present invention is to provide a sensor holder that securely and reliably attaches to a patient's appendage while avoiding excessive vasoconstriction.

Another objective of the present invention is to provide a sensor holder that accommodates an expanded range in patient appendage sizes.

A further objective of the present invention is to provide a sensor holder that has a reduced part count.

One or more of the above objectives and additional advantages are realized by the present invention where a clip-type sensor holder is provided that clips around a portion of a patient appendage without necessarily applying a clamping force to that appendage. In this regard, the sensor holder includes first and second opposing members in an opposing and hinged relationship that allows these members to move from an open position to a closed position relative to one another. When in the closed position, these opposing members are sized to receive a patient appendage (e.g., a finger) therebetween. A resilient member interconnected to each of the members maintains the sensor holder in the closed position about the patient appendage.

According to a first aspect of the present invention, a sensor clip for holding a medical sensor relative to a patient appendage is provided that, when applied to the appendage, applies little or no compressive force thereto. In this regard, the sensor clip includes first and second opposing members that may close about a patient appendage in order to hold a medical sensor relative to that appendage. At least one of these members includes an alignment means for positioning a medical sensor relative to that member. The alignment means allows the medical sensor to be properly positioned in relation to an appendage when the first and second members close about that appendage. Finally, the apparatus includes a resilient means that has a first portion interconnected to the first member and a second portion interconnected to the second member. This resilient member is in a substantially non-biased state when the first and second members are in a closed position relative to one another and disposed about a patient appendage. In this regard, the resilient member is operative to provide a resilient resistive force to movement between the closed and open positions. As will be appreciated in this configuration, the resilient member may apply little or no compressive force to a patient appendage when disposed between the first and second members in a closed position.

In order to receive a patient appendage between the first and second members, one, and more typically, both, of these members may be shaped to conform to such an appendage. For example, when utilized to receive a patient finger, one or both of the first and second members may define a concave surface for conformably receiving the finger. In one embodiment, the first and second members may be sized to substantially surround a patient appendage in the closed position. That is, the first and second members may close around a patient appendage without substantially compressing the appendage.

In order to maintain the opposing members on a patient appendage and to increase patient comfort, a cushion may be integrated onto the inside surfaces of one or both of the first and second members. This cushion(s) may be utilized to create an interference fit between the first and second members and the appendage for retention purposes. As may be appreciated, any compressible material having an adequate compression setting may be utilized to form a cushion. That is, a compressible material (e.g., an open cell foam) operable to conform to a patient appendage without applying substantial pressure to the appendage may be utilized. Furthermore, this cushion may be or comprise a portion of the alignment means for positioning a sensor relative to one or both of the top and bottom members. In this regard, the cushion may be shaped to position a sensor relative to one of the opposing members. For example, the cushion may include a recess sized to receive a portion of a medical sensor. This sensor recess may further include an adhesive (e.g. a peel and stick liner) for attaching the sensor to the apparatus, or, the recess may form a pocket to receive a portion of the sensor. In one embodiment, a recess within the cushion may have a depth substantially the same as the thickness of a medical sensor. As will be appreciated, this allows the surface of the medical sensor and the surface of the cushion to be substantially coplanar when applied to the appendage, thereby preventing the sensor from applying undue pressure to a patient appendage.

Other sensor alignment means may also be utilized. For example, the surfaces of one or both of the opposing members may include protrusions or tabs to align and/or interconnect a sensor to the sensor clip. What is important is that a sensor disposed within the sensor clip remains properly positioned while the sensor clip shuts about a patient appendage.

The hinge member that interconnects the top and bottom members may be any device that allows the first and second members to move between an open and closed position such that a patient appendage may be easily disposed therebetween. In this regard, a separate mechanical hinge may interconnect to the top and bottom members. However, in one embodiment the hinge is integrally formed with the top and bottom members to reduce the part count of the apparatus and to provide for simplified manufacturing.

The resilient member may be any member that elastically deforms under an applied stress and returns to its initial shape after that stress is removed. For example, elastic bands or semi-rigid resilient members (i.e. springs) may be interconnected to the first and second members to provide the resilient resistive force. The resilient member(s) is operative to hold the opposing members shut about a patient appendage. In particular, the resilient member resists movement of the opposing members from a closed position to an open position. This allows the resilient member to maintain the opposing members about an appendage without applying a compressive force to that appendage. In a particular embodiment, the resilient member is substantially non-biased in both the closed and open positions to facilitate placement of an appendage within the sensor clip.

In order for the resilient member to be in a substantially non-biased condition when the sensor clip is in the open and closed position, the distance between the connection points of the resilient member to the opposing members may be substantially equal in the open and closed positions. Accordingly, the geometry of the first and second opposing members may be such that the distance between the connection points increases during at least a portion the movement between the open and closed positions. In this regard, the distance between the connection points may initially increase, reach a maximum value and return to an original length as the hingedly-opposing members move between the open and closed positions, or vise versa.

According to a second aspect of the present invention, a sensor clip is provided for holding a medical sensor relative to a patient appendage wherein the sensor clip is a one-piece unit allowing simplified construction and a reduced part count. Again, the sensor clip has first and second members in an opposing and hinged relationship for opening and closing about a patient appendage. However, the hinge member interconnecting the first and second members is integrally formed with the first and second members in, for example, a molding process in order to provide a one-piece sensor holder. The apparatus also includes a resilient member for holding the first and second opposing members in an open or closed position relative to one another. The resilient member may also be integrally formed with the top and bottom members and the hinge.

The hinge member may be formed as what is commonly referred to as the “living hinge”. In this regard, the hinge member may be a portion of the sensor clip interconnecting the first and second members having reduced cross-sectional area in relation to the first and second members. As will be appreciated, this reduced cross-sectional area allows for concentration of stress therein when bending forces are applied to the first and second members. Accordingly, this hinge member will typically flex prior to the first and second members flexing. The hinge member may comprise one or more thin interconnecting section(s) each having first and second opposing surfaces. Accordingly, these opposing surfaces typically will alternate between compression and tension when the first and second members move between open and closed positions.

The living hinge and the first and second opposing members may be formed in an injection molding process. A variety of materials may be utilized to generate the one-piece injection molded sensor clip. However, it has been found that for most living hinge applications, thermoplastic polymer materials provide improved performance characteristics. That is, thermoplastic polymers allow for increased cycle flexing. Materials utilized for form the one-piece integrally defined sensor clip may be selected from a non-inclusive list including polypropylenes, polyethylenes, and nylons.

According to a third aspect of the present invention, a sensor holder is provided that allows for enhanced sensor placement relative to a patient appendage and increased resistance to accidental removal. In this regard, a clip-type sensor is provided having a hinge axis that is substantially parallel to the insertion direction of the appendage placed therebetween. In this regard, first and second opposing members interconnected by the hinge member may engage a middle or lateral portion of a patient appendage (e.g. a finger may extend through the clip-type sensor). The first and second members may also be sized to substantially surround the lateral portion of the patient appendage. The sensor clip of the subject aspect may also utilize a resilient member to maintain the opposing members in a closed position around the lateral portion of a patient appendage without necessarily applying a compressive force to that appendage.

As will be appreciated, as opposed to a clip-type sensor, which receives a distal end of a patient appendage, this laterally engaging sensor design allows for enhanced placement of a sensor held therein relative to a patient appendage. That is, the sensor holder allows for the sensor to be positioned anywhere along the length of a patient appendage such as a finger and thereby accommodates a wider range of appendage sizes. Furthermore, as the appendage may extend entirely through the sensor clip, the ability of the clip to fall off the appendage is reduced.

According to another aspect of the present invention, a method is provided for holding a medical sensor relative to a patient appendage. The method includes initially biasing first and second opposing members interconnected about a hinge axis in order dispose these members into an open position relative to one another. In particular, the first and second members are biased to overcome a resilient force that resists movement of the members to the open position. Once the first and second opposing members are in the open position, a medical sensor is engaged with at least one of the first and second members. A patient appendage may then be disposed relative to one or both of the first and second members such that, for example, the medical sensor is positioned relative to a desired portion of the appendage. After the medical sensor and appendage are properly positioned relative to the first and second opposing members, those members are again biased about the hinge axis to move the opposing members into a closed position relative to one another. That is, the opposing members may be closed to engage opposing surfaces of the patient appendage and thereby hold the medical sensor relative to that appendage. Again, the first and second members are biased to overcome a resilient force that resists movement of the members from the open position to the closed position.

Biasing the first and second members about a hinge axis typically comprises elastically deforming a resilient member that is interconnected to the first and second opposing members. That is, an elastic band, a spring member or other resilient member(s) may be interconnected to the first and second opposing members and provide resistance to movement between the open and closed positions. Preferably, this resilient member will provide no force (i.e., be substantially non-biased) when the first and second opposing members are in the open and closed positions. In this regard, the first and second opposing members may remain in the open and closed positions without the continued application of force. Biasing the first and second members about the hinge axis may also flex a hinge member interconnecting the first and second members. In this regard, one or more surfaces of the hinge member may move between tensile and compressive states as the first and second members move between open and closed positions.

Disposing the appendage relative to the open sensor clip may comprise engaging such an appendage with a concave surface of one of the opposing members and/or disposing the medical sensor between a portion of the appendage and one of the opposing members. In this regard, a patient appendage may be utilized in part to retain the sensor relative to the sensor holder. In a preferred embodiment, the finger will be disposed relative to the first and second members such that it is substantially parallel to the hinge axis interconnecting those members. As will be appreciated, this allows the first and second members to engage a middle or lateral portion of that patient appendage.

### BRIEF DESCRIPTION OF THE DRAWINGS

FIGS. 1A-1C show three end views of the sensor clip present invention.

FIGS. 2A and 2B show two opposing perspective views of the sensor clip present invention.

FIGS. 3A and 3B show a side and perspective view of the sensor clip applied to a patient appendage.

FIG. 4 shows a sensor that may be utilized with the sensor clip.

FIG. 5 shows the sensor of FIG. 4 disposed within the sensor clip of the present invention.

### DETAILED DESCRIPTION

The present invention will now be described in relation to the accompanying illustrations, which at least assist in illustrating its various pertinent features. The present invention, a sensor holder that clips onto a patient appendage (e.g., finger) is described in conjunction with a pulse oximetry sensor. In particular, the sensor holder is adapted to hold a pulse oximetry sensor having emitters and detectors that are separated such that an optical path is created through patient tissue. However, it will be appreciated that the clip-type sensor holder of the present invention may be utilized with differently configured pulse oximetry sensors as well as other non-invasive medical sensors. In the following description, the invention is set forth in context of an integrally formed sensor holder that utilizes a living hinge member and an “over snap” spring design. This over-snap spring design utilizes a substantially non-biased spring to hold opposing appendage engaging members in a closed position about an appendage (e.g., a finger) to reduce or eliminate pressure applied to the patient's tissue. It will be appreciated, however, that certain aspects of the invention are not limited to such combinations.

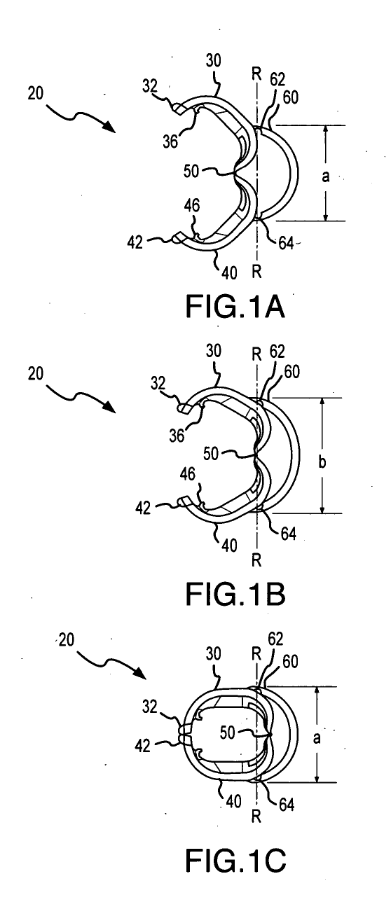

As shown in FIGS. 1A-1C, the clip-type sensor holder 20 (hereinafter “sensor clip”) contains a top member 30 and bottom member 40 interconnected by a hinge 50 that allows the top and bottom members 30, 40 to move between open and closed positions (e.g., to engage a patient appendage). In this regard, the top and bottom members 30, 40 are both trough-shaped in order to receive a patient appendage. The troughs of each member 30, 40 are aligned and define an insertion direction for a patient appendage as well as a longitudinal axis of the sensor clip 20. The clip 20 also includes a spring 60 that is utilized to maintain the top and bottom members 30, 40 in an open or closed relationship relative to one another as will be discussed herein.

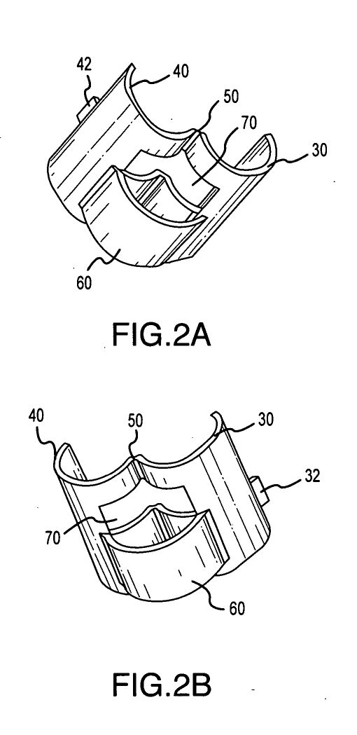

FIGS. 1A-1C show three end views of the sensor clip 20 being moved between an open and a closed position. FIGS. 2A and 2B show opposing perspective views of the sensor clip 20 in the open position. As shown, the sensor clip 20 is formed as a one-piece molded unit. In this regard, the hinge 50 interconnecting the top and bottom members 30, 40 comprises a living hinge formed during a molding process utilized to form the sensor clip 20. As used herein, the term “living hinge” refers to a thin section of a molded unit that connects two segments of that unit and allows those segments to flex repeatedly without the use of a separate mechanical hinge. As will be appreciated, utilization of a living hinge eliminates the need for a separate hinge member to interconnect the top and bottom members 30, 40, thereby providing a simplified sensor holder. As shown in FIGS. 1A-1C, the section of the sensor clip 20 that forms the hinge 50 has a reduced cross-section in comparison to either the top or bottom members 30, 40. Accordingly, this reduced cross-section allows the hinge 50 to flex such that the top and bottom members 30, 40 may move between an open position, FIG. 1A, and a closed position as shown in FIG. 1C.

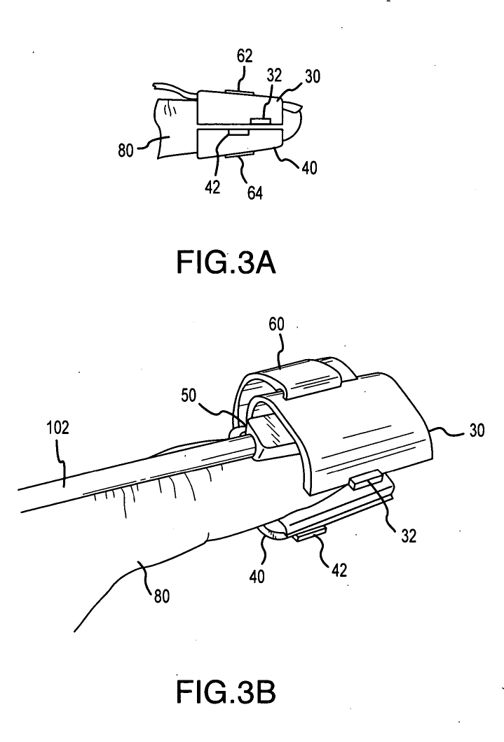

As noted, the top and bottom members, 30, 40 each define a trough that allows these members to receive a patient appendage. In this regard, the top and bottom members 30, 40 may contain troughs having depth, width, and length profiles which are different or substantially the same. What is important, is that the top and bottom members 30, 40 are operable to receive a patient appendage 80 and substantially close around that appendage 80 as shown in FIGS. 3A and 3B. Referring to FIG. 3A, it will be noted that the top and bottom members 30, 40 each contain tapered profile to accommodate for a reduction in patient appendage thickness. As will be appreciated, these top and bottom members 30, 40 may be sized depending on their proposed application. For example, the top and bottom members 30, 40 in a sensor clip 20 intended for use in pediatric applications will be smaller than a similar sensor clip 20 that is intended for use in adult applications. In any case, the top and bottom members 30, 40 are sized such that they substantially enclose about a patient appendage 80 disposed therebetween.

Also incorporated into the molded one-piece sensor clip 20 is a spring 60 for use in maintaining the top and bottom members 30, 40 in either an open position or closed position relative to one another. As shown in FIGS. 1A-1C, the spring 60 has a first end interconnected to top member 30 utilizing a first spring hinge 62, and a second end interconnected to the bottom member 40 utilizing a second spring hinge 64. As with the hinge 50 that interconnects the top and bottom members 30, 40, the spring hinges 62, 64 are living hinges produced during the molding process. Accordingly, these living spring hinges 62, 64 permit flexure between the spring 60 and the top and bottom members 30, 40 when the clip moves between the open and closed positions.

In order to create the one-piece integrally formed sensor clip 20 utilizing living hinges, an injection molding process is utilized. Typically, polymeric materials, which consist of long chains of repeating molecules, are utilized for the injection molding process. In order to create a living hinge member (e.g., 50, 62, 64) these long polymer chains are typically injected in a manner that allows them to be oriented transversely to the hinge axes. To enhance hinge strength, one or both of the following practices may be followed: the injection location for the part (i.e., an injection gate) may be formed to allow the polymers to flow across the hinge axes during injection; and after release from the mold and while still hot, the part may be flexed two or more times to strengthen the hinges. Materials particularly apt for use in creating living hinges include: thermoplastic polymers such as polyethylenes, polypropylenes, and nylons. As will be appreciated, these materials may each be tailored to provide various desired characteristics. For example, dyes may be readily utilized with polypropylenes to produce sensor clips 20 having any of a variety of colors. That is, the sensor clip 20 may be made opaque for light-blocking characteristics. Alternatively, the sensor clip 20 may be made substantially transparent such that a sensor may be held relative to its outside surfaces for use in monitoring a patient appendage disposed relative to an inside surface of the sensor clip.

As noted, the integrally formed spring 60 is operative to maintain the top and bottom members 30, 40 in either open or closed positions. Due to the geometry of the sensor clip 20, the spring 60 is substantially non-biased when the top and bottom members 30, 40 are open, and when they are closed. In this regard, the spring 60 resists movement between the two positions, but does not necessarily provide any compressive force when the top and bottom members 30, 40 are closed. Referring to FIGS. 1A-1C, this spring orientation is more fully described. As shown in FIG. 1A, the spring 60 is in a neutral, non-biased position holding the top and bottom members 30, 40 in the open position. In the open position, the spring 60 has a first length (a) between the first and second spring hinges 62, 64. Likewise, in FIG. 1C when the top and bottom members 30, 40 are in the closed position the spring 60 again has the first length (a) between the first and second spring hinges 62, 64. In contrast, when the top and bottom members 30, 40 are moved between the open and closed positions, the spring 60 is biased to a second length (b) and thereby provides a resistance force to this change in position.

By way of example, to close the sensor clip 20, a compressive force is applied to the top and bottom members 30, 40. This compressive force rotates these members 30, 40 about the hinge 50. As shown in FIG. 1A, the hinge 50 is initially disposed to the left of a reference axis R-R passing through the spring hinges 62, 64. As the top and bottom members 30, 40 continue rotating from an open to closed position, the hinge axis 50 moves to the right and intersects the reference axis R-R between the spring hinges 62, 64. See FIG. 1B. Accordingly, the distance between the spring hinges 62 and 64 is expanded to a second length (b) to allow the hinge 50 to pass therebetween. This expansion biases the spring 60 providing a resistance to this motion. As the top and bottom members 30, 40 continue to close, the hinge 50 is disposed to the right of the reference axis R-R. Accordingly, the distance between the spring hinges 62, 64 returns to the first length (a) as the hinge 50 moves further to the right returning the spring 60 to a non-biased position as shown in FIG. 1C. That is, the spring 60 snaps over the top and bottom members 30, 40 and holds them in a closed position. While the top and bottom members 30, 40 are in the closed position, the non-biased spring 60 may provide substantially no compressive force therebetween. In this regard, when positioned on a patient appendage 80, substantially no compressive force may be applied to the appendage 80 which may cause, for example, pressure necrosis. However, the spring resists the opening of the opposing top and bottom members from 30, 40.

Since the spring 60 does not apply a compressive force for maintaining the sensor clip 20 on a patient appendage 80, other means may be utilized to maintain the sensor clip 20 on the appendage 80. For example, the inside surface of one or both of the top and bottom members may include a compressive foam material. When positioned on the appendage 80, this compressive foam material forms a cushion 90 that provides an interference fit between the sensor clip 20 and the appendage 80. See FIG. 5.

Utilization of a side hinge configuration where the sensor clip 20 snaps shut around a patient appendage 80 may also provide for improved sensor clip 20 retention. As shown in FIGS. 3A and 3B, the hinge 50 interconnecting the top and bottom members 30, 40 is substantially parallel to the patient appendage, thereby allowing the sensor clip 20 to be engaged around a lateral portion of a patient appendage 80. That is, as opposed to a clip-type sensor holder that engages the end of an appendage, the present embodiment of the sensor clip 20 engages around a lateral portion of an appendage 80, thereby reducing the likelihood of unintended sensor clip removal.

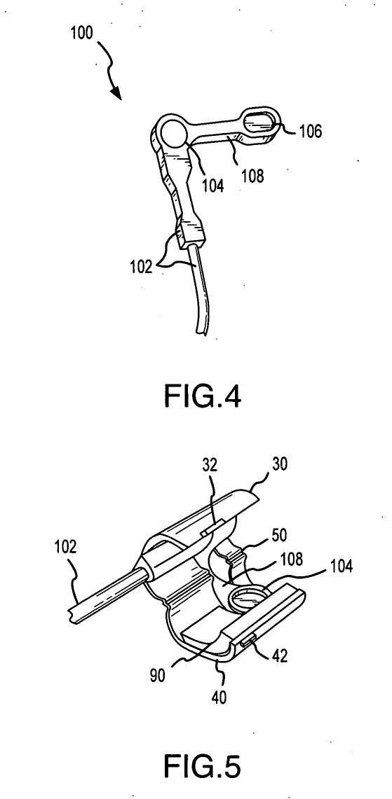

An added benefit of this side-hinge design is the increased flexibility in sensor positioning relative to a patient appendage 80. That is, a sensor clip 20 that engages an end portion of a patient finger may only be able to dispose the finger a limited distance between an emitter 104 and detector 106 of a sensor 100 (see e.g., FIG. 4), as may be limited by the depth of the sensor clip 20 and/or the flexible conduit 108 interconnecting the emitter 104 and detector 106. In contrast, where the emitter 104 and detector 106 are held on the top and bottom members 30, 40 of the sensor clip 20 utilizing a side hinge 50, and the flexible conduit 108 is disposed around a side portion of the appendage 80, the sensor clip 20 may be moved to multiple positions along the length of the patient appendage 80. As will be appreciated, this allows for enhanced sensor placement that may allow for improved blood analyte computations.

As shown, the sensor clip 20 is utilized to hold a pulse oximetry sensor relative to a patient appendage. Such a sensor is shown in FIG. 4. As shown, the sensor 100 is a reusable sensor that may be disinfected between uses on different patients. In this regard, the sensor clip 20 may be a disposable unit that is used on a single patient then disposed for sanitation purposes. However, this is not a requirement. The sensor 100 includes a connection cable 102, at least one light emitter 104, a light detector 106, and a flexible wiring conduit 108 interconnecting the detector 106 and emitter 104. As will be appreciated, the signal connection cable 102 may be interconnected to a pulse oximetry monitor which provides drive signals to effect light emission by the light emitters 104 and which processes detection signals output by the detectors 106 in order to calculate blood analyte concentrations. As shown, the sensor 100 is a transmittance-type pulse oximetry sensor having a light emitter 104 and light detector 106 designed to be held on opposing surfaces of a patient appendage 80 to create an optical path through living tissue. Accordingly, the sensor clip 20 is sized to receive the sensor 100 such that the emitter 104 and detector 106 are held adjacent to the patient appendage 80 when the top and bottom members 30, 40 are closed.

FIG. 3B shows a perspective view of the sensor clip 20 holding the pulse oximetry sensor 100 relative to the patient appendage 80. In this regard, the cable 102 and the light emitter(s) 104 are held between the top member 30 of the sensor clip 20 and the patient appendage 80. Likewise, the light detector 106 is held between the patient appendage 80 and the bottom member 40. Of note, the top and bottom members 30, 40 in combination form an aperture 70 sized to receive the flexible conduit 80 interconnecting the light emitter 104 and light detector 106. FIG. 5 shows the sensor 100 disposed within the sensor clip 20 prior to application to a patient appendage. To facilitate sensor placement within the sensor clip 20 includes sensor retaining tabs 36, 46 on the top and bottom members 30, 40. These retaining tabs 36, 46 are sized and positioned to engage opposing ends of the sensor 100 thereby preventing sensor movement while the top and bottom members 30, 40 rotate shut. As the opposing ends of the sensor 100 are prevented from moving the flexible conduit 108 bends during this movement and is disposed through the aperture 70.

Though the embodiments discussed above utilize a disposable sensor clip 20 with a reusable sensor 100, it will be appreciated that various aspects of the present invention may be otherwise utilized. For example, a substantially similar sensor clip may be provided that contains a built-in pulse oximetry sensor. In this regard, light emitter(s) and detector(s) may be incorporated (e.g. adhered) onto a surface of the top and bottom members 30. 40. Furthermore, the top and bottom members 30, 40 may be formed with apertures in their surfaces such that the emitter(s) and detector(s) may be disposed on an outside surface of the sensor clip 20. Alternatively, the sensor clip 20 could be over-molded onto the reusable sensor 100 as described above.

The embodiments described above are for exemplary purposes only and is not intended to limit the scope of the present invention. Various adaptations, modifications and extensions of the embodiment will be apparent to those skilled in the art and are intended to be within the scope of the invention as defined by the claims that follow.

1. An apparatus for holding a medical sensor relative to a patient appendage, comprising:
first and second members in an opposing and hinged relationship, said first and second members being selectively movable between an open position and a closed position;
an alignment means associated with one of said first and second members for positioning a medical sensor relative to one of said first and second members; and
a resilient member having a first portion interconnected to said first member and a second portion interconnected to said second member, wherein said resilient member is in a substantially non-biased state when said first and second members are in said open and closed positions and wherein said resilient member resists movement of said first and second members between said open and closed position.

2. The apparatus as recited in claim 1, further comprising:
a hinge member interconnecting said first and second members.

3. The apparatus as recited in claim 2, wherein said hinge member and said first and second members are an integrally defined one-piece unit.

4. The apparatus as recited in claim 2, wherein said hinge member, said first and second members, and said resilient member are an integrally defined one-piece unit.

5. The apparatus as recited in claim 2, wherein said hinge member defines a hinge axis extending substantially parallel to a longitudinal center axis of said sensor holder.

6. The apparatus as recited in claim 5, wherein said hinge axis is substantially parallel to a direction of patient appendage insertion between first and second members.

7. The apparatus as recited in claim 1, wherein at least one of said first and second members is configured to define a concave surface for receiving a patient appendage.

8. The apparatus as recited in claim 7, wherein said first and second members are configured to receive an end portion of a patient appendage.

9. The apparatus as recited in claim 7, wherein said concave surface has a center axis extending substantially parallel to a hinge axis interconnecting said first and second members.

10. The apparatus as recited in claim 9, wherein said first and second members are configured to substantially surround a middle portion of a patient appendage.

11. The apparatus as recited in claim 1, wherein at least one of said first and second members further comprises:
a cushion interconnected to a patient appendage interface surface.

12. The apparatus as recited in claim 11, wherein said alignment means comprises:
a recess at least partially formed within said cushion for receiving at least a portion of a medical sensor.

13. The apparatus as recited in claim 12, wherein a depth of said recess is substantially equal to the thickness of said sensor.

14. The apparatus as recited in claim 1, wherein said alignment means comprise at least one of:
a projection on a patient engaging surface of one of said first and second members, said projection for engaging a medical sensor; and
a recess on a patient engaging surface of one of said first and second members, said recess sized to receive at least a portion of a medical sensor.

15. The apparatus as recited in claim 1, wherein said alignment means comprises an aperture extending through one of said first and second members, said aperture allowing access between first and second members when said first and second members are in said closed position.

16. The apparatus as recited in claim 1, wherein said resilient member comprises a semi-rigid member.

17. The apparatus as recited in claim 16, wherein said semi-rigid resilient member is hingedly interconnected to said first and second members.

18. The apparatus as recited in claim 1, further comprising at least one of:
a light emitter mounted to one of said first and second members for emitting light; and
a light detector mounted to one of said first and second members for detecting light and providing a signal indicative of said detected light.

19. The apparatus as recited in claim 18, wherein at least one of said light emitter and said light detector is disposed between said first and second members when said first and second members are in said closed position.

20. A medical sensor holder for holding a medical sensor relative to a patient's appendage, comprising:
first and second appendage engaging members in an opposing relationship; said members being selectively movable between open and closed positions; and
an alignment means associated with one of said first and second members for positioning a medical sensor relative to one of said first and second members; and
a hinge member interconnecting said first and second members, wherein first and second portions of said hinge member are in tension and compression, respectively, in said open position and in compression and tension in said closed position, respectively;
wherein said first and second members and said hinge member are an integrally defined one-piece unit.

21. The sensor holder of claim 20, wherein said integrally defined one-piece unit comprises a polymer material.

22. The sensor holder of claim 21, wherein said integrally defined one-piece unit is of molded construction.

23. The sensor holder of claim 21, wherein said polymer material is selected from a group consisting of:
polypropylenes;
polyethylenes; and
nylons.

24. The sensor holder of claim 20, further comprising:
a resilient member having a first portion interconnected to said first member and a second portion interconnected to said second member for providing a resilient force to maintain said first and second members in one of said open and closed positions.

25. The sensor holder as recited in claim 24, wherein said resilient member is integrally defined with said first and second members and said hinge member.

26. The sensor holder as recited in claim 20, wherein said hinge member defines a hinge axis extending substantially parallel to a longitudinal center axis of said sensor holder.

27. The sensor holder as recited in claim 26, wherein said hinge axis is substantially parallel to a direction of patient appendage insertion between said first and second members.

28. The sensor holder as recited in claim 20, wherein at least one of said first and second members is configured to define a concave surface for receiving a patient appendage.

29. The sensor holder as recited in claim 28, wherein said first and second members are configured to substantially surround a portion of a patient appendage in said closed position.

30. The sensor holder as recited in claim 29, wherein said first and second members are configured to substantially surround a middle portion of a patient appendage in said closed position.

31. The sensor holder as recited in claim 20, wherein said resilient member is in a substantially non-biased state when said first and second members are in said closed position.

32. The sensor holder as recited in claim 31, wherein said resilient member provides a resilient resistance force for opposing movement from said closed position to said open position.

33. A medical sensor holder for holding a medical sensor relative to a patient appendage, comprising:
first and second members in an opposing relationship to which a medical sensor may be selectively connected;
a hinge member interconnecting said first and second members and defining a hinge axis, said hinge axis being substantially parallel to a direction of patient appendage insertion between said first and second members;
wherein said first and second members are configured to engage a middle portion of a patient appendage in said closed position.

34. The sensor holder as recited in claim 33, wherein said first and second members are configured to, in combination, substantially surround a middle portion of a patient appendage in said closed position.

35. The sensor holder as recited in claim 33, wherein said first and second members and said hinge member are an integrally defined one-piece unit.

36. The sensor holder as recited in claim 33, further comprising:
a resilient member having a first portion interconnected to said first member and a second portion interconnected to said second member for maintaining said first and second members in one of an open position and a closed position relative to one another.

37. The sensor holder as recited in claim 36, wherein said first and second members, said hinge member, and said resilient member are an integrally defined one-piece unit.

38. The sensor holder as recited in claim 36, wherein said resilient member is in a substantially non-biased state when first and second members are in said closed position.

39. The sensor holder as recited in claim 38, wherein said resilient member provides a resilient force to oppose movement from said closed position to said open position.

40. The apparatus as recited in claim 33, further comprising at least one of:
a light emitter mounted to one of said first and second members for emitting light; and
a light detector mounted to one of said first and second members for detecting light and providing a signal indicative of said detected light.

41. A method for holding a sensor relative to a patient appendage, comprising:
first biasing first and second interconnected members about a hinge axis to overcome a resilient resistive force and dispose said members from a closed position into an open position;
engaging a medical sensor with one of said first and second members;
disposing a patient appendage relative to at least one of said first and second members; and
second biasing said first and second members about said hinge axis to overcome said resilient resistive force in order to dispose said members form said open position to said closed position, wherein said appendage is disposed between said first and second members and said medical sensor is held relative to said appendage.

42. The method of claim 41, wherein disposing said appendage step comprises disposing said appendage relative to one of said first and second members wherein said appendage is substantially parallel with said hinge axis.

43. The method of claim 41, wherein disposing said appendage step comprises disposing a middle portion of said appendage relative to said first and second members.

44. The method of claim 41, wherein said first and second biasing steps comprise biasing a resilient member from a non-biased condition to a biased condition to allow said first and second members to move into said open and closed positions, respectively.

45. The method of claim 44, wherein said resilient member is in a substantially non-biased condition when said first and second members are in said open and closed positions.

46. The method of claim 41, wherein moving between said open and closed positions flexes a hinge member interconnecting said first and second members.

47. The method of claim 46 wherein opposing surfaces of said hinge member are in tension and compression, respectively, in said open position and said opposing surfaces are in compression and tension, respectively, in said closed position.
